SEQUENCE LISTING (2 sequences)

SEQ ID NO: 1 - DNA, 43 bp, Artificial Sequence
  primer
gatcggggcccagccggccaccttgaactcagttctcatcaat

SEQ ID NO: 2 - DNA, 79 bp, Artificial Sequence
  primer
gatcggatcctcaatggtgatggtgatgatgcttgtcatcgtcgtccttgtagtcgtgtctctggcaggtgtggagcct

(19) United States
(12) Patent Application Publication
(10) Pub. No.: US 2005/0169995 A1
(43) Pub. Date: Aug. 4, 2005

(54) Modulation of gastrointestinal epithelium proliferation through the Wnt signaling pathway

(76) Inventor: Calvin Jay Kuo, Stanford, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/958,531

(22) Filed: Oct. 4, 2004

Related U.S. Application Data
(60) Provisional application No. 60/508,346, filed on Oct. 3, 2003.

(51) Int. Cl. A61K 9/64; A61K 9/24; A61K 38/17; A61K 48/00

(52) U.S. Cl. 424/472; 514/2; 514/44

(57) ABSTRACT

These results indicate the efficacy of systemic expression of secreted Wnt antagonists as a general strategy for conditional inactivation of Wnt signaling in adult organisms, and illustrate a striking reliance on a single growth factor pathway for the maintenance of the architecture of the adult small intestine and colon. These results also indicate the potential utility of administration of Wnt pathway agonists for mucosal repair of the small intestine or colon.

The adult intestinal epithelium is characterized by continuous replacement of epithelial cells through a stereotyped cycle of cell division, differentiation, migration and exfoliation occurring during a 5-7 day crypt-villus transit time. The putative growth factors regulating proliferation within the adult intestinal stem cell niche have not yet been identified, although studies have implicated the cell-intrinsic action of β-catenin/Lef/Tcf signaling within the proliferative crypt compartment.

A number of pathological conditions affect the cells of the intestines. Inflammatory bowel disease (IBD) can involve either or both the small and large bowel. Crohn's disease and ulcerative colitis are the best-known forms of IBD, and both fall into the category of “idiopathic” inflammatory bowel disease because the etiology for them is unknown. “Active” IBD is characterized by acute inflammation. “Chronic” IBD is characterized by architectural changes of crypt distortion and scarring. Crypt abscesses can occur in many forms of IBD.

Ulcerative colitis (UC) involves the colon as a diffuse mucosal disease with distal predominance. The rectum is virtually always involved, and additional portions of colon may be involved extending proximally from the rectum in a continuous pattern. The etiology for UC is unknown. Patients with prolonged UC are at increased risk for developing colon cancer. Patients with UC are also at risk for development of liver diseases including sclerosing cholangitis and bile duct carcinoma.

Crohn's disease can involve any part of the GI tract, but most frequently involves the distal small bowel and colon. Inflammation is typically transmural and can produce anything from a small ulcer over a lymphoid follicle (aphthoid ulcer) to a deep fissuring ulcer to transmural scarring and chronic inflammation. One third of cases have granulomas, and extracolonic sites such as lymph nodes, liver, and joints may also have granulomas. The transmural inflammation leads to the development of fistulas between loops of bowel and other structures. Inflammation is typically segmental with uninvolved bowel separating areas of involved bowel. The etiology is unknown, though infectious and immunologic mechanisms have been proposed.

Gluten, a common dietary protein present in wheat, barley and rye causes a disease called Celiac Sprue in sensitive individuals. Ingestion of such proteins by sensitive individuals produces flattening of the normally luxurious, rug-like, epithelial lining of the small intestine. Other clinical symptoms of Celiac Sprue include fatigue, chronic diarrhea, malabsorption of nutrients, weight loss, abdominal distension, anemia, as well as a substantially enhanced risk for the development of osteoporosis and intestinal malignancies such as lymphoma and carcinoma. Celiac Sprue is generally considered to be an autoimmune disease and the antibodies found in the serum of the patients support the theory that the disease is immunological in nature.

In contrast to these conditions where malabsorption of nutrients may be found, obese patients may deliberately seek to reduce their digestive ability. Human obesity is a widespread and serious disorder, affecting a high percentage of the adult population in developed countries. In spite of an association with heart disease, type II diabetes, cancer, and other conditions, few persons are able to permanently achieve significant weight loss. Failure to treat obesity may be at least partially attributed to the complexity of the disease. Genetic, psychological and environmental factors all play a role in individual patterns of weight gain or loss, making it exceedingly difficult to define the contribution of any single element.

Wnt proteins form a family of highly conserved secreted signaling molecules that regulate cell-to-cell interactions during embryogenesis. Wnt genes and Wnt signaling are also implicated in cancer. Insights into the mechanisms of Wnt action have emerged from several systems: genetics in Drosophila and Caenorhabditis elegans; biochemistry in cell culture and ectopic gene expression in Xenopus embryos. Many Wnt genes in the mouse have been mutated, leading to very specific developmental defects. As currently understood, Wnt proteins bind to receptors of the Frizzled family on the cell surface. Through several cytoplasmic relay components, the signal is transduced to beta-catenin, which then enters the nucleus and forms a complex with TCF to activate transcription of Wnt target genes. Expression of Wnt proteins varies, but is often associated with developmental process, for example in embryonic and fetal tissues.

The exploration of physiologic functions of Wnt proteins in adult organisms has been hampered by functional redundancy and the necessity for conditional inactivation strategies. Dickkopf-1 (Dkk1) has been recently identified as the founding member of a family of secreted proteins that potently antagonize Wnt signaling (see Glinka et al. (1998) Nature 391:357-62; Fedi et al. (1999) J Biol Chem 274:19465-72; and Bafico et al. (2001) Nat Cell Biol 3:683-6). Dkk1 associates with both the Wnt co-receptors LRP5/6 and the transmembrane protein Kremen, with the resultant ternary complex engendering rapid LRP6 internalization and impairment of Wnt signaling through the absence of functional Frizzled/LRP6 Wnt receptor complexes Mao et al. (2001) Nature 411:321-5; Semenov et al. (2001) Curr Biol 11:951-61; and Mao et al. (2002) Nature 417:664-7.

Transgenic mice that have a knock-out of the Tcf locus show a loss of proliferative stem cell compartments in the small intestine during late embryogenesis. However, the knockout is lethal, and so has not been studied in adults. In chimeric transgenic mice that allow analysis of adults, expression of constitutively active NH₂-truncated β-catenin stimulated proliferation in small intestine crypts, although either NH₂-truncated β-catenin or Lef-1/b-catenin fusions induced increased crypt apoptosis as well. Because diverse factors regulate β-catenin/Lef/Tcf-dependent transcription, including non-Frizzled GPCRs and PTEN/PI-3-kinase, the cause of intestinal stem cell defect is not known.

Developing pharmacologic agents for the regulation of intestinal epithelium growth is of great interest for clinical purposes. The present invention addresses this issue.

### SUMMARY OF THE INVENTION

Methods are provided for modulating the growth of intestinal epithelial cells, through alterations in the wnt signaling pathway. Wnts are shown to be essential growth factors required for maintenance of the robust proliferation characteristic of both the adult small and large intestine.

In one embodiment of the invention, inhibitors of wnt signaling are administered for short-term reduction of intestinal epithelial proliferation in the treatment of obesity. Such a reduction is shown to result in long term weight loss.

In another embodiment of the invention, activators of wnt signaling are administered to enhance proliferation of intestinal epithelium, for the treatment, or as a therapeutic adjunct in the treatment, of diseases that compromise the intestinal epithelia, including inflammatory bowel diseases and celiac sprue.

### BRIEF DESCRIPTION OF THE DRAWINGS

FIGS. 1A-1E. Analysis of adenoviruses expressing murine Dkk1. (A) Construction of Ad Dkk1. Murine Dkk1 cDNA bearing N-terminal IgK signal peptide and C-terminal FLAG and 6×His tags was inserted into E1-E3-adenovirus strain 5 by homologous recombination. (B) Inhibition of Wnt3a-stimulated TOPFLASH luciferase reporter activity by transfected Dkk1. Wnt3a and/or Dkk1 expression vectors were co-transfected into 293T cells with pTOPFLASH as described in methods followed by luciferase measurement. (C). Beta-catenin stabilization assay. Purified recombinant Dkk1 (125 ng/ml) was added to L cells 2 hours prior to recombinant Wnt3A (1:8000, Nusse Laboratory), followed after 3 hours by harvest and Western blot analysis for b-catenin (BD Transduction Labs). (D) Time course of Dkk1 expression in the circulation. Adult C57BI/6 mice received single i.v. tail vein injection of Ad Dkk1 (10⁹ pfu) followed by phlebotomy at the indicated times. Dkk1 was detected by Western blotting using anti-His probe Ab (Santa Cruz) and migrated as a doublet of 38/34 kDa. (E) Survival analysis of C57BI/6 mice following Ad Dkk1 (IgK signal/3′ FLAG/His), Ad Dkk1-HA (IgK signal/5′HA/3′ FLAG/His) or Ad Fc treatment. 10⁹ pfu were administered i.v. unless otherwise indicated. Ad Fc and Ad Dkk1-HA 3×10⁸ and 1×10⁸ pfu doses have not exhibited any mortality over a 120-day time course.

FIG. 2. Time course of histological changes in the gastrointestinal tract of Ad Dkk1-treated animals. Adult C57BI/6J mice (12-16 week old) received single i.v. tail vein injection of 10⁹ pfu of either Ad Dkk1 or the negative control virus Ad Fc (10⁹ pfu) followed by analysis of organs by H&E staining at the indicated times. Dkk1 induced architectural changes in small intestine characterized by crypt loss followed by villus blunting and fusion, loss of mucosal integrity and mucosal regeneration by day 10, demonstrated by large basophilic crypts in duodenum and jejunum. The stomach was relatively unaffected. In cecum and colon, Dkk1 induced crypt loss with profound mucosal degeneration and ulceration by day 7 and regeneration by day 10 evidenced by irregular basophilic crypts at day 10. Stomach (st), duodenum (du), proximal jejunum (je), ileum (il). cecum (ce), colon (co).

FIG. 3. Spectrum of colonic lesions in Ad Fc- or Ad Dkk1 -treated C57BI/6J (top panel) and SCID (bottom panel) mice. Colons were harvested for H&E staining at day 7 after adminstration of 10⁹ pfu of the appropriate adenoviruses. Moderate thinning of the ascending colon in C57BI/6J mice versus frequent ulceration in SCID mice is depicted. A spectrum of lesions was observed in descending colons of both C57BI/6J and SCID mice, ranging from focal ulceration to frank effacement of architecture and replacement with inflammatory infiltrates, the latter being less severe in SCID animals.

FIG. 4. Expression analysis of Wnt/β-catenin target genes CD44 and EphB2 in the gastrointestinal tract of Ad Dkk1- or Ad Fc-treated adult C57BI/6 mice (12-16 week old). Organs were harvested 2 days after virus administration. (Left panels) Ad Dkk1 repression of CD44 expression in proliferative zones of all levels of the gastrointestinal epithelium. CD44 immunohistochemistry was performed as described in the text. Arrowheads indicate the absence of CD44 expression in the proliferative compartments of the intestinal epithelium in Ad Dkk1 animals. Asterisks denote residual CD44 staining in the non-epithelial lamina propria. (Right panels) Ad Dkk1 repression of EphB2 expression in small intestine and colon. Strong repression of EphB2 was observed in the small intestine, cecum and descending colon. Weaker repression was found in the ascending colon, while EphB2 expression appeared largely unaffected in the stomach. EphB2 immunofluorescence was performed with Alexa488 detection of EphB2 immunoreactivity (green) and Hoechst nuclear counterstain (blue). Stomach (st), duodenum (du), jejunum (je), ileum (il), cecum (ce), ascending colon (ac), descending colon (dc).

FIG. 5. Analysis of proliferative state in the stem cell compartments of the gastrointestinal epithelium at day 2 in Ad Dkk1-treated C57BI/6J mice by Ki67 immunohistochemistry. Arrowheads indicate the absence of Ki67 in the proliferative compartments of the gastrointestinal epithelium in Ad Dkk1 animals. Note the strong repression of Ki67 immunoreactivity in duodenum, jejunum, cecum and descending colon, and moderate reduction in ascending colon. Ileum and stomach were not significantly affected.

FIG. 6. Intestinal epithelial differentiation is not affected in Ad Dkk1 treated animals.

Representative sections of the duodenum of Ad Fc or Ad Dkk1 treated mice on day two after virus application were analyzed for the presence of differentiated intestinal epithelial cell types. Top panels: FABP-L immunohistochemistry identifies absorptive enterocytes. Middle panels: Alcian blue staining for secretory goblet cells is combined with anti-Lysozyme immunohistochemistry (brown precipitate) marking Paneth cells. Bottom panels: Gremelius staining for enteroendocrine cells. Rare positive cells are indicated by arrowheads.

FIG. 7. Analysis of apoptotic state in the stem cell compartments of the gastrointestinal epithelium at day 2 in Ad Dkk1-treated animals by TUNEL staining. Arrowheads indicate rare positive apoptotic cells. No increase in apoptisis was detected in Ad Dkk1 compared to Ad Fc mice. Additional positive TUNEL staining was observed in the villus tips but did not vary between Ad Dkk1 and Ad Fc.

FIG. 8. Prolonged weight loss after low dose Ad Dkk injection. Adult C57BI/6 mice (12 weeks old) were given a single injection of the Ad DKK vector at 3×10⁸ pfu, or 10⁹ PFU.

Weight was monitored for a period of greater than 50 days.

### DESCRIPTION OF THE SPECIFIC EMBODIMENTS

The administration of agents that activate or inhibit wnt is used to modulate the proliferation of cells in the intestinal epithelium. Inhibition of proliferation results in dramatic weight loss, which loss is shown to be maintained for an extended period of time. Activation of proliferation is useful in the treatment of diseases that compromise the intestinal epithelia, including inflammatory bowel diseases and celiac sprue.

### DEFINITIONS

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

Wnt polypeptide and agonists thereof As used herein, the terms “Wnts” or “Wnt gene product” or “Wnt polypeptide” when used herein encompass native sequence Wnt polypeptides, Wnt polypeptide variants, Wnt polypeptide fragments and chimeric Wnt polypeptides. In some embodiments of the invention, the Wnt protein comprises palmitate covalently bound to a cysteine residue. A “native sequence” polypeptide is one that has the same amino acid sequence as a Wnt polypeptide derived from nature. The native sequence of human Wnt polypeptides may range from about 348 to about 389 amino acids long in their unprocessed forms, reflecting variability at the poorly conserved amino-terminus and several internal sites, contain 21 conserved cysteines, and have the features of a secreted protein. The molecular weight of a Wnt polypeptide is usually about 38-42 kD.

The term “native sequence Wnt polypeptide” includes human Wnt polypeptides. Human wnt proteins include the following: Wnt 1, Genbank reference NP_{—}005421.1; Wnt 2, Genbank reference NP_{—}003382.1, which is expressed in brain in the thalamus, in fetal and adult lung and in placenta; two isoforms of Wnt 2B, Genbank references NP_{—}004176.2 and NP_{—}078613.1. Isoform 1 is expressed in adult heart, brain, placenta, lung, prostate, testis, ovary, small intestine and colon. In the adult brain, it is mainly found in the caudate nucleus, subthalamic nucleus and thalamus. Also detected in fetal brain, lung and kidney. Isoform 2 is expressed in fetal brain, fetal lung, fetal kidney, caudate nucleus, testis and cancer cell lines. Wnt 3 and Wnt3A play distinct roles in cell-cell signaling during morphogenesis of the developing neural tube, and have the Genbank references NP_{—}110380.1 and X56842. Wnt3A is expressed in bone marrow. Wnt 4 has the Genbank reference NP_{—}110388.2. Wnt 5A and Wnt 5B have the Genbank references NP_{—}003383.1 and AK013218. Wnt 6 has the Genbank reference NP_{—}006513.1; Wnt 7A is expressed in placenta, kidney, testis, uterus, fetal lung, and fetal and adult brain, Genbank reference NP_{—}004616.2. Wnt 7B is moderately expressed in fetal brain, weakly expressed in fetal lung and kidney, and faintly expressed in adult brain, lung and prostate, Genbank reference NP_{—}478679.1. Wnt 8A has two alternative transcripts, Genbank references NP_{—}114139.1 and NP_{—}490645.1. Wnt 8B is expressed in the forebrain, and has the Genbank reference NP_{—}003384.1. Wnt 10A has the Genbank reference NP_{—}079492.2. Wnt 10B is detected in most adult tissues, with highest levels in heart and skeletal muscle. It has the Genbank reference NP_{—}003385.2. Wnt 11 is expressed in fetal lung, kidney, adult heart, liver, skeletal muscle, and pancreas, and has the Genbank reference NP_{—}004617.2. Wnt 14 has the Genbank reference NP_{—}003386.1. Wnt 15 is moderately expressed in fetal kidney and adult kidney, and is also found in brain. It has the Genbank reference NP_{—}003387.1. Wnt 16 has two isoforms, Wnt-16a and Wnt-16b, produced by alternative splicing. Isoform Wnt-16B is expressed in peripheral lymphoid organs such as spleen, appendix, and lymph nodes, in kidney but not in bone marrow. Isoform Wnt-16a is expressed at significant levels only in the pancreas. The Genbank references are NP_{—}057171.2 and NP_{—}476509.1.

Other activators of wnt signaling include compounds that bind to, and activate receptors of the Frizzled family on the cell surface, e.g. antibodies and fragments thereof, wnt mimetics and derivatives, and the like. An additional method of achieving Wnt inhibition is the neutralization of a Wnt inhibitor, i.e. the chelation of Dkk by a soluble ectodomain of Kremen1/2 or LRP5/6).

Casein kinase Iε (CKIε) has been identified as a positive regulator of the Wnt signaling pathway, for example see Peters et al. (1999) Nature 401:345-350; and Sakanaka et al. (1999) Proc. Natl. Acad. Sci. USA 96:12548-12552. The α isoform of CKI is, in contrast, a negative regulator of Wnt signaling, by functioning as a priming kinase for β-catenin and GSK3. Inhibitors of casein kinase are known, and include, for example 3-[(2,4,6-trimethoxyphenyl)methylidenyl]-indolin-2-one (IC261) (Mashhoon et al. (2000) J. Biol. Chem. 275:20052-20060.

GSK3β is one of the components of a protein complex that regulates the stability of β-catenin. Phosphorylation of the GSK3β sites in the N terminus of β-catenin is believed to be a signal for degradation. GSK3β has been placed between Dishevelled and β-catenin in the Wnt pathway (Hooper et al. (1994) Nature 372:461-464; Siegfried et al. (1994) Nature 367:76-80). Inhibition of GSK3β activity by lithium salt or GSK3β-binding protein (GBP/FRAT) mimics Wnt signaling. GSK3b inhibitors are known in the art, for examples see Kelly et al. (2004) Exp Neurol. 188(2):378-86; Wan et al. (2004) Chem Biol. 11 (2):247-59; Bhat et al. (2003) J Biol Chem. (2003) 278(46):45937-45; and Wagman et al. (2004) Curr Pharm Des. 10(10): 1105-37.

Wnt inhibitor. Wnt inhibitors are agents that downregulate expression or activity of wnt. Agents of interest may interact directly with wnt, e.g. blocking antibodies, or may interact with wnt associated proteins,. e.g. Wnt co-receptors LRP5/6 and the transmembrane protein Kremen. A number of wnt inhibitors have been described and are known in the art, including those described above.

Among the known wnt inhibitors are members of the Dickkopf (Dkk) gene family (see Krupnik et al. (1999) Gene 238(2):301 -13). Members of the human Dkk gene family include Dkk-1, Dkk-2, Dkk-3, and Dkk-4, and the Dkk-3 related protein Soggy (Sgy). hDkks 1-4 contain two distinct cysteine-rich domains in which the positions of 10 cysteine residues are highly conserved between family members. Exemplary sequences of human Dkk genes and proteins are publicly available, e.g. Genbank accession number NM_{—}014419 (soggy-1); NM_{—}014420 (DKK4); AF177394 (DKK-1); AF177395 (DKK-2); NM_{—}015881 (DKK3); and NM_{—}014421 (DKK2).

Inhibitors may also include derivatives, variants, and biologically active fragments of Dkk polypeptides. A “variant” polypeptide means a biologically active polypeptide as defined below having less than 100% sequence identity with a native sequence polypeptide. Such variants include polypeptides wherein one or more amino acid residues are added, at the N- or C-terminus of, or within, the native sequence; from about one to forty amino acid residues are deleted, and optionally substituted by one or more amino acid residues; and derivatives of the above polypeptides, wherein an amino acid residue has been covalently modified so that the resulting product has a non-naturally occurring amino acid. Ordinarily, a biologically active variant will have an amino acid sequence having at least about 90% amino acid sequence identity with a native sequence polypeptide, preferably at least about 95%, more preferably at least about 99%.

A “chimeric” Dkk polypeptide is a polypeptide comprising a polypeptide or portion (e.g., one or more domains) thereof fused or bonded to heterologous polypeptide. The chimeric Wnt polypeptide will generally share at least one biological property in common with a native sequence Wnt polypeptide. Examples of chimeric polypeptides include immunoadhesins, combine a portion of the Dkk polypeptide with an immunoglobulin sequence, and epitope tagged polypeptides, which comprise a Dkk polypeptide or portion thereof fused to a “tag polypeptide”. The tag polypeptide has enough residues to provide an epitope against which an antibody can be made, yet is short enough such that it does not interfere with biological activity of the Dkk polypeptide. Suitable tag polypeptides generally have at least six amino acid residues and usually between about 6-60 amino acid residues.

A “functional derivative” of a native sequence Dkk polypeptide is a compound having a qualitative biological property in common with a native sequence Dkk polypeptide. “Functional derivatives” include, but are not limited to, fragments of a native sequence and derivatives of a native sequence Dkk polypeptide and its fragments, provided that they have a biological activity in common with a corresponding native sequence Dkk polypeptide. The term “derivative” encompasses both amino acid sequence variants of Dkk polypeptide and covalent modifications there

Other inhibitors of wnt include Wise (Itasaki et al. (2003) Development 130(18):4295-30), which is a secreted protein. The Wise protein physically interacts with the Wnt co-receptor, lipoprotein receptor-related protein 6 (LRP6), and is able to compete with Wnt8 for binding to LRP6. Axin regulates Wnt signaling through down-regulation of beta-catenin (see Lyu et al. (2003) J Biol Chem. 278(15): 13487-95).

A soluble form of the ligand binding domain (CRD) of Frizzled has also been shown to inhibit wnt. The Frizzled-CRD domain has been shown to inhibit the Wnt pathway by inhibiting the binding of Wnts to the frizzled receptor (Hsieh et al. (1999) Proc Natl Acad Sci U S A 96:3546-51; and Cadigan et al. (1998) Cell 93:767-77). Polypeptides of interest include FRP5, FRP8, and the like. Similarly, SFRPs represent secreted molecules which encode Frizzled-like CRDs and thus represent soluble Wnt antagonists by functioning as soluble receptors (Krypta et al, J Cell Sci Jul. 1, 2003;116(Pt 13):2627-34).

Compound screening. Candidate modulators of wnt signaling may be identified by detecting the ability of an agent to affect the biological activity of wnt, as described below. A plurality of assays may be run in parallel with different concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in binding.

Compounds of interest for screening include biologically active agents of numerous chemical classes, primarily organic molecules, although including in some instances inorganic molecules, organometallic molecules, genetic sequences, etc. Candidate agents comprise functional groups necessary for structural interaction with proteins, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Compounds are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Molecules of interest as activators and inhibitor include specific binding members that bind to, e.g. wnt, frizzled, wnt co-receptors, and the like. The term “specific binding member” or “binding member” as used herein refers to a member of a specific binding pair, i.e. two molecules, usually two different molecules, where one of the molecules (i.e., first specific binding member) through chemical or physical means specifically binds to the other molecule (i.e., second specific binding member). Specific binding pairs of interest include carbohydrates and lectins; complementary nucleotide sequences; peptide ligands and receptor; effector and receptor molecules; hormones and hormone binding protein; enzyme cofactors and enzymes; enzyme inhibitors and enzymes; lipid and lipid-binding protein; etc. The specific binding pairs may include analogs, derivatives and fragments of the original specific binding member.

In a preferred embodiment, the specific binding member is an antibody. The term “antibody” or “antibody moiety” is intended to include any polypeptide chain-containing molecular structure with a specific shape that fits to and recognizes an epitope, where one or more non-covalent binding interactions stabilize the complex between the molecular structure and the epitope. Antibodies utilized in the present invention may be polyclonal antibodies, although monoclonal antibodies are preferred because they may be reproduced by cell culture or recombinantly, and can be modified to reduce their antigenicity.

Polyclonal antibodies can be raised by a standard protocol by injecting a production animal with an antigenic composition. See, e.g., Harlow and Lane, Antibodies: A Laboratory Manual, Cold Spring Harbor Laboratory, 1988. When utilizing an entire protein, or a larger section of the protein, antibodies may be raised by immunizing the production animal with the protein and a suitable adjuvant (e.g., Fruend's, Fruend's complete, oil-in-water emulsions, etc.) When a smaller peptide is utilized, it is advantageous to conjugate the peptide with a larger molecule to make an immunostimulatory conjugate. Commonly utilized conjugate proteins that are commercially available for such use include bovine serum albumin (BSA) and keyhole limpet hemocyanin (KLH). In order to raise antibodies to particular epitopes, peptides derived from the full sequence may be utilized. Alternatively, in order to generate antibodies to relatively short peptide portions of the brain tumor protein target, a superior immune response may be elicited if the polypeptide is joined to a carrier protein, such as ovalbumin, BSA or KLH. Alternatively, for monoclonal antibodies, hybridomas may be formed by isolating the stimulated immune cells, such as those from the spleen of the inoculated animal. These cells are then fused to immortalized cells, such as myeloma cells or transformed cells, which are capable of replicating indefinitely in cell culture, thereby producing an immortal, immunoglobulin-secreting cell line. In addition, the antibodies or antigen binding fragments may be produced by genetic engineering. Humanized, chimeric, or xenogenic human antibodies, which produce less of an immune response when administered to humans, are preferred for use in the present invention.

In addition to entire immunoglobulins (or their recombinant counterparts), immunoglobulin fragments comprising the epitope binding site (e.g., Fab′, F(ab′)₂, or other fragments) are useful as antibody moieties in the present invention. Such antibody fragments may be generated from whole immunoglobulins by ficin, pepsin, papain, or other protease cleavage. “Fragment,” or minimal immunoglobulins may be designed utilizing recombinant immunoglobulin techniques. For instance “Fv” immunoglobulins for use in the present invention may be produced by linking a variable light chain region to a variable heavy chain region via a peptide linker (e.g., poly-glycine or another sequence which does not form an alpha helix or beta sheet motif).

Wnt modulation. The methods of the present invention utilize inhibition or activation of wnt signaling. In general, the effect of the agents on intestinal epithelium will be indicative of the wnt activity. Such activity may be monitored by histological analysis, expression of wnt/β-catenin target genes; measurement of proliferation in stem cell compartments; and the like. For example, inhibition of wnt may result in crypt loss followed by villus blunting and fusion and loss of mucosal integrity. Genes expressed in the gastrointestinal tract that are controlled by wnt/β-catenin include CD44, and EphB2. Antibodies specific for these proteins are commercially available. Analysis of proliferation may utilize staining for Ki67, which is a nuclear protein expressed in proliferating cells during late G1-, S—, M-, and G2-phases of the cell cycle, while cells in the G0 (quiscent) phase are negative.

For screening purposes one may utilize in vitro assays for wnt biological activity, e.g. stabilization of β-catenin, promoting growth of stem cells, etc. Assays for biological activity of Wnt include stabilization of p-catenin, which can be measured, for example, by serial dilutions of the Wnt composition. An exemplary assay for Wnt biological activity contacts a Wnt composition in the presence of a candidate inhibitor or activator with cells, e.g. mouse L cells. The cells are cultured for a period of time sufficient to stabilize β-catenin, usually at least about 1 hour, and lysed. The cell lysate is resolved by SDS PAGE, then transferred to nitrocellulose and probed with antibodies specific for β-catenin.

### Delivery of Wnt Modulating Agent

The wnt modulating agents are incorporated into a variety of formulations for therapeutic administration. In one aspect, the agents are formulated into pharmaceutical compositions by combination with appropriate, pharmaceutically acceptable carriers or diluents, and are formulated into preparations in solid, semi-solid, liquid or gaseous forms, such as tablets, capsules, powders, granules, ointments, solutions, suppositories, injections, inhalants, gels, microspheres, and aerosols. As such, administration can be achieved in various ways, usually by oral administration. The agent may be systemic after administration or may be localized by virtue of the formulation, or by the use of an implant that acts to retain the active dose at the site of implantation.

In pharmaceutical dosage forms, the wnt modulating agent and/or other compounds may be administered in the form of their pharmaceutically acceptable salts, or they may also be used alone or in appropriate association, as well as in combination with other pharmaceutically active compounds. The agents may be combined to provide a cocktail of activities. The following methods and excipients are exemplary and are not to be construed as limiting the invention.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

In one embodiment of the invention, the oral formulations comprise enteric coatings, so that the active agent is delivered to the intestinal tract. Enteric formulations are often used to protect an active ingredient from the strongly acid contents of the stomach. Such formulations are created by coating a solid dosage form with a film of a polymer that is insoluble in acid environments, and soluble in basic environments. Exemplary films are cellulose acetate phthalate, polyvinyl acetate phthalate, hydroxypropyl methylcellulose phthalate and hydroxypropyl methylcellulose acetate succinate, methacrylate copolymers, and cellulose acetate phthalate.

Other enteric formulations comprise engineered polymer microspheres made of biologically erodable polymers, which display strong adhesive interactions with gastrointestinal mucus and cellular linings and can traverse both the mucosal absorptive epithelium and the follicle-associated epithelium covering the lymphoid tissue of Peyer's patches. The polymers maintain contact with intestinal epithelium for extended periods of time and actually penetrate it, through and between cells. See, for example, Mathiowitz et al. (1997) Nature 386 (6623): 410-414. Drug delivery systems can also utilize a core of superporous hydrogels (SPH) and SPH composite (SPHC), as described by Dorkoosh et al. (2001) J Control Release 71 (3):307-18.

In another embodiment, a microorganism, for example adenovirus, bacterial or yeast culture, capable of producing wnt modulating polypeptide is administered to a patient. Such a culture may be formulated as an enteric capsule; for example, see U.S. Pat. No. 6,008,027, incorporated herein by reference. Alternatively, microorganisms stable to stomach acidity can be administered in a capsule, or admixed with food preparations.

Other formulations of interest include formulations of DNA encoding agents of interest, so as to target intestinal cells for genetic modification. For example, see U.S. Pat. No. 6,258,789, herein incorporated by reference, which discloses the genetic alteration of intestinal epithelial cells.

Formulations are typically provided in a unit dosage form, where the term “unit dosage form,” refers to physically discrete units suitable as unitary dosages for human subjects, each unit containing a predetermined quantity of glutenase in an amount calculated sufficient to produce the desired effect in association with a pharmaceutically acceptable diluent, carrier or vehicle. The specifications for the unit dosage forms of the present invention depend on the particular complex employed and the effect to be achieved, and the pharmacodynamics associated with each complex in the host.

The pharmaceutically acceptable excipients, such as vehicles, adjuvants, carriers or diluents, are commercially available. Moreover, pharmaceutically acceptable auxiliary substances, such as pH adjusting and buffering agents, tonicity adjusting agents, stabilizers, wetting agents and the like, are commercially available. Any compound useful in the methods and compositions of the invention can be provided as a pharmaceutically acceptable base addition salt. “Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Those of skill will readily appreciate that dose levels can vary as a function of the specific enzyme, the severity of the symptoms and the susceptibility of the subject to side effects. Some of the agents will be more potent than others. Preferred dosages for a given agent are readily determinable by those of skill in the art by a variety of means. A preferred means is to measure the physiological potency of a given compound.

### Therapeutic Methods

The subject methods are useful for both prophylactic and therapeutic purposes. Thus, as used herein, the term “treating” is used to refer to both prevention of disease, and treatment of a pre-existing condition. The treatment of ongoing disease, to stabilize or improve the clinical symptoms of the patient, is a particularly important benefit provided by the present invention. Such treatment is desirably performed prior to loss of function in the affected tissues; consequently, the prophylactic therapeutic benefits provided by the invention are also important. Evidence of therapeutic effect may be any diminution in the severity of disease. The therapeutic effect can be measured in terms of clinical outcome or can be determined by immunological or biochemical tests.

Various methods for administration may be employed, preferably using oral administration, for example with meals. The dosage of the therapeutic formulation will vary widely, depending upon the nature of the disease, the frequency of administration, the manner of administration, the clearance of the agent from the host, and the like. The initial dose can be larger, followed by smaller maintenance doses. The dose can be administered as infrequently as weekly or biweekly, or more often fractionated into smaller doses and administered daily, with meals, semi-weekly, or otherwise as needed to maintain an effective dosage level.

For the treatment of obesity, inhibitors of wnt signaling are administered at a dose that is effective to cause short term dimunition of intestinal epithelial cell proliferation, but which maintains the overall health of the individual. The treatment regime can require administration for prolonged periods, but may be administered as a single dose monthly, semi-monthly, etc. The size of the dose administered must be determined by a physician and will depend on a number of factors, such as the nature and gravity of the disease, the age and state of health of the patient and the patient's tolerance to the drug itself.

In a specific embodiment, the wnt inhibitor can be used for treatment of obese patients by means of a short-term (1-2 weeks) administration, in order to obtain a rapid, significant decrease in body weight (5-10%), which can be maintained subsequently using an appropriate diet and/or physical exercise.

Patients may use various criteria for determining obesity. Conveniently, a body mass index (BMI) is calculated, where a person having a BMI of greater than 25 is overweight and may considered for treatment with the subject methods. There is a high degree of interrelationship between obesity and type II (adult onset) diabetes. Patients suitable for the treatment with the subject inhibitors include those with diabetes. This condition can be life-threatening, and high glucose levels in the blood plasma (hyperglycemia) can lead to a number of chronic diabetes syndromes, for example, atherosclerosis, microangiopathy, kidney disorders, renal failure, cardiac disease, diabetic retinopathy and other ocular disorders including blindness.

For the treatment of diseases where upregulation of intestinal epithelium is desired, enhancers of wnt signaling, e.g. soluble wnt protein, agonists of frizzled receptor, inhibitors of Wnt inhibitors (i.e. soluble Kremen or LRP/5/6 to chelate Dkk) and the like are administered. The therapy may be combined with other methods of treatment, e.g. immune suppression, diet, etc.

Celiac sprue is typically diagnosed through clinical symptoms, including fatigue, chronic diarrhea, malabsorption of nutrients, weight loss, abdominal distension, and anemia. Other disease indicia include the presence of antibodies specific for glutens, antibodies specific for tissue transglutaminase, the presence of pro-inflammatory T cells and cytokines, and degradation of the villus structure of the small intestine. Application of the methods and compositions of the invention can result in the improvement of any and all of these disease sumptoms.

Wnt and wnt agonists are also administered for the treatment of gastrointestinal inflammation. “Gastrointestinal inflammation” as used herein refers to inflammation of a mucosal layer of the gastrointestinal tract, and encompasses acute and chronic inflammatory conditions. Acute inflammation is generally characterized by a short time of onset and infiltration or influx of neutrophils.

“Chronic gastrointestinal inflammation” refers to inflammation of the mucosal of the gastrointestinal tract that is characterized by a relatively longer period of onset, is long-lasting (e.g., from several days, weeks, months, or years and up to the life of the subject), and is associated with infiltration or influx of mononuclear cells and can be further associated with periods of spontaneous remission and spontaneous occurrence. Thus, subjects with chronic gastrointestinal inflammation may be expected to require a long period of supervision, observation, or care. “Chronic gastrointestinal inflammatory conditions” (also referred to as “chronic gastrointestinal inflammatory diseases”) having such chronic inflammation include, but are not necessarily limited to, inflammatory bowel disease (IBD), colitis induced by environmental insults (e.g., gastrointestinal inflammation (e.g., colitis) caused by or associated with (e.g., as a side effect) a therapeutic regimen, such as administration of chemotherapy, radiation therapy, and the like), colitis in conditions such as chronic granulomatous disease (Schappi et al. Arch Dis Child. 2001 February;1984(2):147-151), celiac disease, celiac sprue (a heritable disease in which the intestinal lining is inflamed in response to the ingestion of a protein known as gluten), food allergies, gastritis, infectious gastritis or enterocolitis (e.g., Helicobacter pylori-infected chronic active gastritis) and other forms of gastrointestinal inflammation caused by an infectious agent, and other like conditions.

As used herein, “inflammatory bowel disease” or “IBD” refers to any of a variety of diseases characterized by inflammation of all or part of the intestines. Examples of inflammatory bowel disease include, but are not limited to, Crohn's disease and ulcerative colitis. Reference to IBD throughout the specification is often referred to in the specification as exemplary of gastrointestinal inflammatory conditions, and is not meant to be limiting.

Wnt or wnt agonists can be administered to a subject prior to onset of more severe symptoms (e.g., prior to onset of an acute inflammatory attack), or after onset of acute or chronic symptoms (e.g., after onset of an acute inflammatory attack). As such, the agents can be administered at any time, and may be administered at any interval. In one embodiment, wnt or wnt agonists are administered about 8 hours, about 12 hours, about 24 hours, about 2 days, about 4 days, about 8 days, about 16 days, about 30 days or 1 month, about 2 months, about 4 months, about 8 months, or about 1 year after initial onset of gastrointestinal inflammation-associated symptoms and/or after diagnosis of gastrointestinal inflammation in the subject.

When multiple doses are administered, subsequent doses are administered within about 16 weeks, about 12 weeks, about 8 weeks, about 6 weeks, about 4 weeks, about 2 weeks, about 1 week, about 5 days, about 72 hours, about 48 hours, about 24 hours, about 12 hours, about 8 hours, about 4 hours, or about 2 hours or less of the previous dose. In one embodiment, ISS are administered at intervals ranging from at least every two weeks to every four weeks (e.g., monthly intervals) in order to maintain the maximal desired therapeutic effect (e.g., to provide for maintenance of relief from IBD-associated symptoms).

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Experimental

Adenoviral expression of Dkk1 (Ad Dkk1) is used to achieve stringent, fully conditional and reversible Wnt inhibition in transgenic adult mice.

Methods

Ad Construction and Production. Dkk1 cDNA was amplified from embryonic day (E)17.5 mouse embryo cDNA with C-terminal FLAG and/or His6 epitope tags, sequenced, and cloned into the E1 region of E1⁻E3⁻ Ad strain 5 by homologous recombination, followed by Ad production in 293 cells and CsCl gradient purification of virus as previously described. The negative control virus Ad Fc expressing a murine IgG2a Fc fragment has been described.

Ad Administration and Detection of Plasma Transgene Expression. Adult (12-16 weeks old) male C57BL/6 or CB17 severe combined immunodeficient (SCID) mice received single i.v. tail vein injection of 10⁹ pfu of the appropriate Ads. For low-dose studies, 3×10⁸ plaque-forming units (pfu) were administered. At appropriate times after injection, whole blood was obtained by retroorbital phlebotomy followed by Western blot analysis of 1 μl of plasma using anti-His probe antibody (Santa Cruz Biotechnology) or anti-His C-term antibody (Invitrogen). Low-dose (3×10⁸) administration was estimated to produce 10-20% of the circulating Dkk1 levels in high-dose animals (10⁹ pfu).

Immunohistochemistry and Histology. The following antibodies were used: Rat anti-mouse CD44 (1:100; BD Pharmingen), rat anti-mouse Ki67 (1:100; DAKO), goat anti-mouse EphB2 (1:100; R & D Systems), rabbit anti-rat FABP (1:100; Novus Biologicals, Littleton, CO), and rabbit anti-human lysozyme (1:100; DAKO). Immunostainings of paraffin-embedded samples were performed according to standard procedures. Antigen retrieval was accomplished by boiling samples in Na-citrate buffer (10 mM, pH 6.0) for 20 min. Color development was performed by using diaminobenzidine (brown precipitate) with hematoxylin counterstain. For immunofluorescence, samples were cryoembedded in OCT compound and sectioned at 7-μM thickness. Stainings were visualized with Alexa 488-conjugated secondary anti-goat antibodies (Molecular Probes) and nuclei were counterstained with Hoechst 33342 (Molecular Probes). For histological analysis, hematoxylin/eosin and Alcian blue staining of paraffin-embedded sections was performed according to standard protocols. Gremelius staining was performed by using Pascual's modified method. Terminal deoxynucleotidyltransferase-mediated dUTP nick end labeling (TUNEL) staining on paraffin-embedded samples used 20 μM biotin-16-UTP and 0.4 units/μl terminal transferase followed by color development (Vectastain ABC kit, Vector Laboratories) and methyl green counterstaining.

Construction of Dkk1 Ads. Dkk1 cDNA was amplified from E17.5 mouse embryo cDNA by PCR, using the forward primer (SEQ ID NO:1) 5′-GAT CGG GGC CCA GCC GGC CAC CTT GAA CTC AGT TCT CAT CAA T-3′ and the reverse primer (SEQ ID NO:2) 5′-GAT CGG ATC CTC AAT GGT GAT GGT GAT GAT GCT TGT CAT CGT CGT CCT TGT AGT CGT GTC TCT GGC AGG TGT GGA GCC T-3′, which incorporated C-terminal FLAG and His₆ epitope tags. The PCR product was cloned into pCR2.1 (Invitrogen), was sequenced and was subcloned SfiI-SalI as an in-frame fusion with the IgK signal peptide downstream of the human CMV promoter of the Ad shuttle plasmid, Add2 SecTag, a variant of Add2. For murine Dkk1-HA containing an N-terminal HA and C-terminal FLAG and His₆ epitope tags, the Dkk1A insert was excised SfiI-SalI and ligated in-frame into SfiI-SalI-cut Ad shuttle plasmid Add2 Display, a variant of Add2 containing a 5′ IgK signal peptide and an HA tag. The Dkk1 and Dkk1-HA inserts were cloned into the E1 region of E1-E3-Ad strain 5 as using homologous recombination, followed by Ad production in 293 cells and CsCl gradient purification of virus. The negative control virus Ad Fc expressing a murine IgG2a Fc fragment has been described by Kuo et al. (2001) Proc. Natl. Acad. Sci. USA 98, 4605-4610.

β-catenin Stabilization Assay. L cells were grown in DMEM containing 10% FBS and seeded in 24-well plates at a density of 2×10⁵ cells per well. The cells were treated with 125 ng/ml Dkk1 purified over Ni-agarose from adenoviral supernatant for 2 h, after which purified Wnt3a protein was added for an additional 3 h (1:8,000). Cells were washed in PBS and lysed in TNT buffer (150 mM NaCl/50 mM Tris-HCl, pH 7.5/1% Triton X-100). The cell lysates were analyzed for β-catenin levels by using Western blotting and anti-β-catenin mAb (BD Transduction Laboratories, Stanford, Calif.).

Luciferase Reporter Assays. The 293T cells were seeded in 24-well plates at a density of 1×10⁵ cells per well. Plasmids transfected are as follows (μ g per well): pTOPFLASH, 0.1; EF-LacZ, 0.1; PGKWnt3a, 0.3; Add Dkk1, 0.3. Total DNA transfected was normalized to 0.8 μg per well by using PGK vector. Luciferase assays were performed using the Dual-Light reporter gene assay system (Tropix, Bedford, Mass.). Luciferase activity was normalized against β-galactosidase activity and all assays were performed in triplicate.

Quantitation of Proliferative Index. Ki67-positive epithelial cells were quantitated on 3-5 high-powered fields for each portion of the gastrointestinal tract. Fields were selected for similar tissue planes and an equivalent number of anatomic structures (e.g., villi) were analyzed on each field. The observer was blinded to the treatment conditions of the mice.

Results

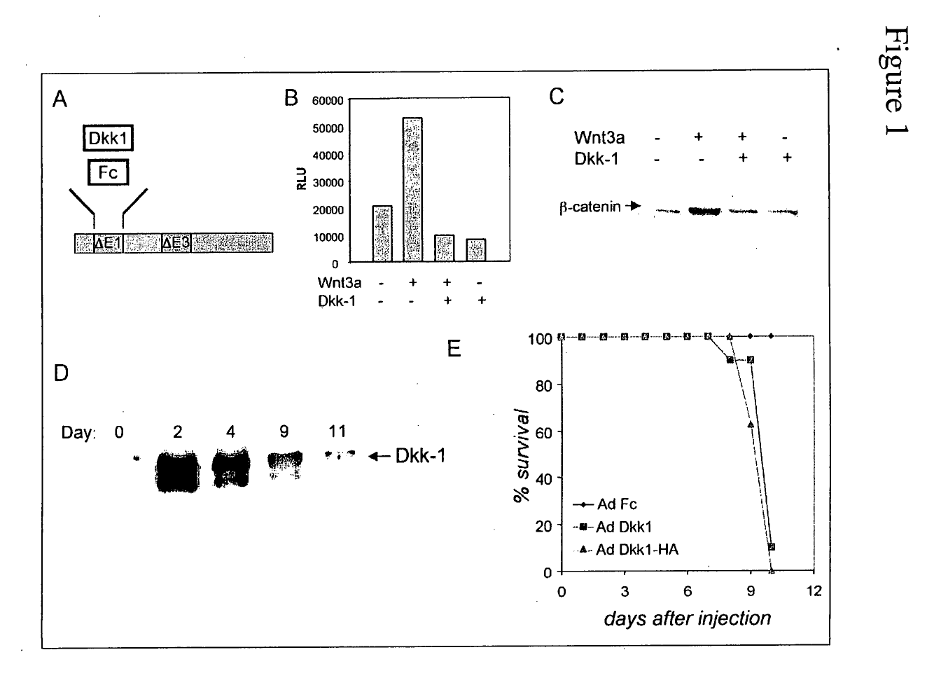

To achieve conditional Wnt inactivation in adult animals, an Ad-expressing murine Dkk1 cDNA bearing C-terminal His6 and Flag epitope tags was produced (Ad Dkk1) by conventional methods (FIG. 1A). The transfected adenoviral Dkk1 shuttle plasmid inhibited Wnt3a-stimulated transcription of a TOPFLASH reporter gene (FIG. 1B), whereas recombinant Dkk1 purified from Ad Dkk1 supernatants inhibited recombinant Wnt3a-induced -catenin stabilization in L cells (FIG. 1C), which is consistent with appropriate functional activity. Single i.v. injection of purified Ad Dkk1 (10⁹ pfu) into tail veins of adult (12-16 weeks old) C57BU6 mice resulted in liver transduction and produced transient Dkk1 expression in plasma peaking at day 2 and progressively diminishing over an 11-day period (FIG. 1D), which is in agreement with the typical expression kinetics of Ads in immunocompetent mice.

Single i.v. administration of Ad Dkk1 (10⁹ pfu) to adult C57BU6 mice produced progressive weight loss and frequent melena or hematochezia with >85% mortality by 10 days (FIG. 1E). An identical phenotype was observed with an independent Ad expressing an N-terminal hemagglutinin (HA)-tagged Dkk1 (Ad Dkk1 -HA) (FIG. 1E). In contrast, significant weight loss, gastrointestinal bleeding, or mortality were not observed with control Ads expressing either an Ig IgG2 Fc fragment (Ad Fc) (FIG. 1E), the non-Wnt inhibitor Dkk3, or the soluble VEGF receptor, Flk1-Fc, at levels comparable to, or exceeding that of, Ad Dkk1. Ad Dkk1 doses of 3×10⁸ pfu or lower produced progressively less precipitous weight loss and were not associated with either hematochezia, melena, or mortality over a 120-day time course.

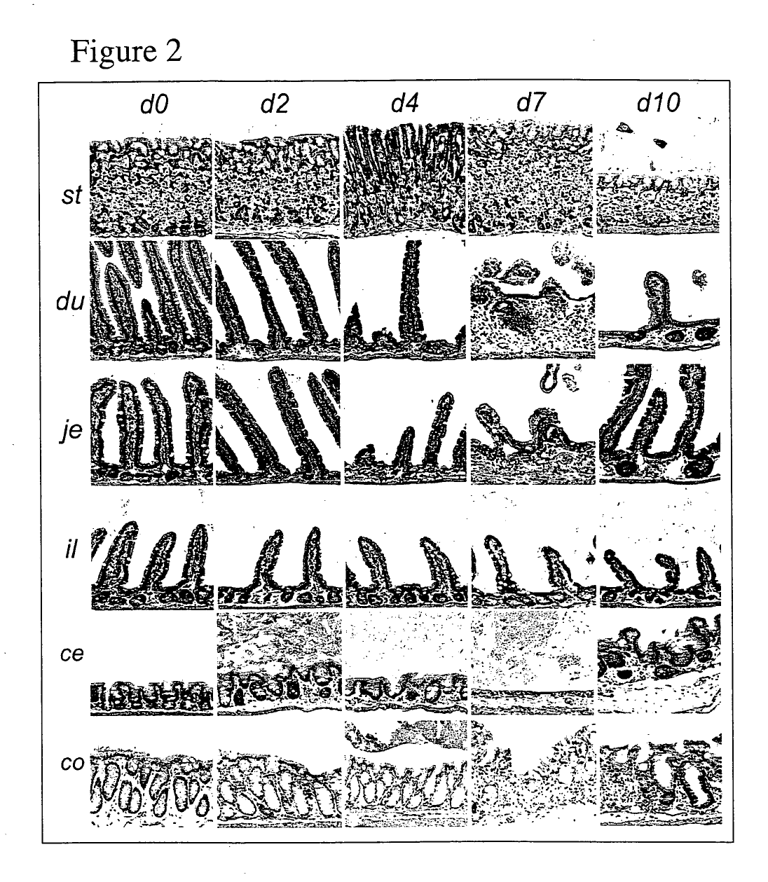

The ease of preparation of Ad combined with the convenience of single-injection dosing facilitated examination of synchronized cohorts of Ad Dkk1-treated animals (10⁹ pfu) over defined intervals of a 10-day time course. Mucosal architecture in duodenum and proximal jejunum was severely distorted with rapid and near-total loss of crypts and decreased villus density by days 2 and 4, without inflammation or crypt necrosis (FIG. 2). In remnant crypts, Paneth cells predominated, and, by day 7, crypt loss was followed by villus blunting and fusion, loss of mucosal integrity, and frank ulceration and mucosal hemorrhage with mixed inflammatory infiltrate in the lamina propria. The small intestine exhibited a proximal-distal gradient of histologic effects with most severe phenotypes observed in duodenum and proximal jejunum, with the distal jejunum and ileum manifesting only mild crypt loss and villus blunting (FIG. 2).

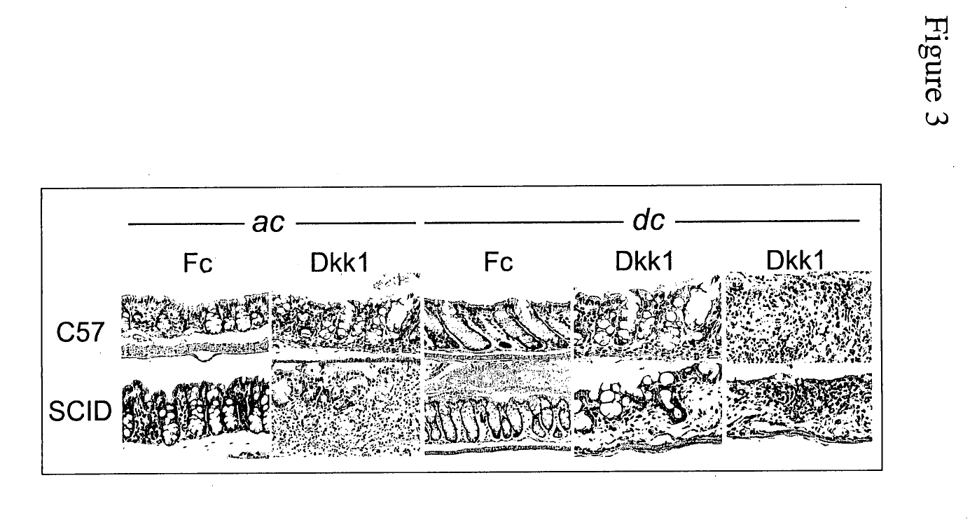

In the colon and cecum of C57BL/6J mice, only mild glandular thinning and/or crypt loss was observed at days 2 and 4, which was in contrast to striking crypt loss and villus blunting in the small intestine (FIG. 2). However, by day 7, the cecal and colonic epithelium exhibited multifocal mucosal degeneration and ulceration of a severity exceeding that of the small intestine, with the descending colon more severely affected than the ascending colon (FIG. 2). The spectrum of colonic lesions ranged from noninvolved foci particularly in ascending colon, to mild glandular thinning, focal ulceration, and extensive areas with complete effacement of architecture and replacement with mixed inflammatory infiltrates (FIG. 3). Ad Dkk1 treatment of CB17 SCID mice lacking B and T lymphocytes resulted in an identical spectrum of colon architectural lesions as in C57BL/6J mice, suggesting that the observed colitis in C57BU6J mice was not inflammatory or autoimmune in nature. However, the ascending colon was more severely affected in SCID than C57 with more extensive and ulcerated lesions (FIG. 3), which was potentially consistent with higher level and more persistent adenoviral gene expression in immunocompromised SCID mice. Similarly, rectums of Ad Dkk1-treated SCID mice exhibited frequent ulceration as opposed to mild glandular thinning in C57BL/6J mice. In contrast to the profound changes in small intestine and colon, the stomach of both strains exhibited only moderate glandular thinning at late time points that could not be distinguished from gastric atrophy secondary to inappetance (FIG. 2). Ad Dkk1 small intestine phenotypes were identical in both C57BL/6J and SCID mice, with severe involvement of duodenum and jejunum and notable absence of pathology in ileum. A summary table of gastrointestinal phenotypes in C57BU6J and SCID mice is presented in Table 1.

TABLE 1
Summary of severity and penetrance
of gastrointestinal phenotypes induced by Ad Dkk1

| | C57BL/6 | | SCID | |
|---|---|---|---|---|
| | Severity | Penetrance | Severity | Penetrance |
| Stomach | − | 8/8 | − | 6/6 |
| Duodenum | +++ | 11/12 | +++ | 8/8 |
| Jejunum | +++/++ | 12/12 | +++ | 8/8 |
| Ileum | + | 6/12 | + | 7/8 |
| Cecum | +++ | 9/9 | +++ | 6/6 |
| Ascending colon | ++ | 8/9 | +++ | 5/6 |
| Descending colon | +++ | 9/9 | +++ | 6/6 |

−, unaffected.
+, minimal changes; e.g., increase in individual necrotic cells, mild villus blunting.
++, moderate changes, typically moderate reduction in crypt/gland numbers without other changes or mild multifocal ulceration in a background of healthy hyperplastic mucosa.
+++, severe changes, typically severe ulceration with associated inflammation, with or without hyperplasia.

Animals treated with lower doses of Ad Dkk1 (3×10⁸ pfu) exhibited 80% lower plasma levels and displayed a less severe intestinal phenotype relative to high-dose (10⁹ pfu) animals, illustrating dose dependency of Ad Dkk1. In these lower-dose animals, decreased small intestine crypt density with overall intact mucosal architecture was observed at day 4 in duodenum but not jejunum and ileum. In cecum and colon of low-dose animals, ulceration, edema, and inflammation were less severe than with high dose, and these animals did not exhibit mortality over a 120-day time course.

In both small and large intestine, decreased adenoviral transgene expression at day 10 (FIG. 1D) was accompanied by epithelial regeneration, which was consistent with a reversible effect. By day 10, duodenum and jejunum exhibited small numbers of regenerative basophilic, hyperplastic crypts, with more advanced reconstitution of villus structure in jejunum than duodenum (FIG. 2). In day 10 colon, hyperplastic regenerative crypts coexisted with persistent multifocal mucosal ulceration (FIGS. 2 and 3). Despite this regenerative response, frequent mortality was observed with high doses of Ad Dkk1 (10⁹ pfu) at days 8-10, which was likely secondary to colitis and systemic infection, with elevated WBC counts (>20×10³/μl) and a left-shifted differential commonly noted in premorbid mice. Examination of adherens junctions in nonulcerated areas by electron microscopy and by immunofluorescence did not reveal significant alterations, whereas histologic examination of other solid organs including liver revealed them to be unaffected in a Dkk1 -specific fashion, except for thymic atrophy, which could not be distinguished from systemic illness.

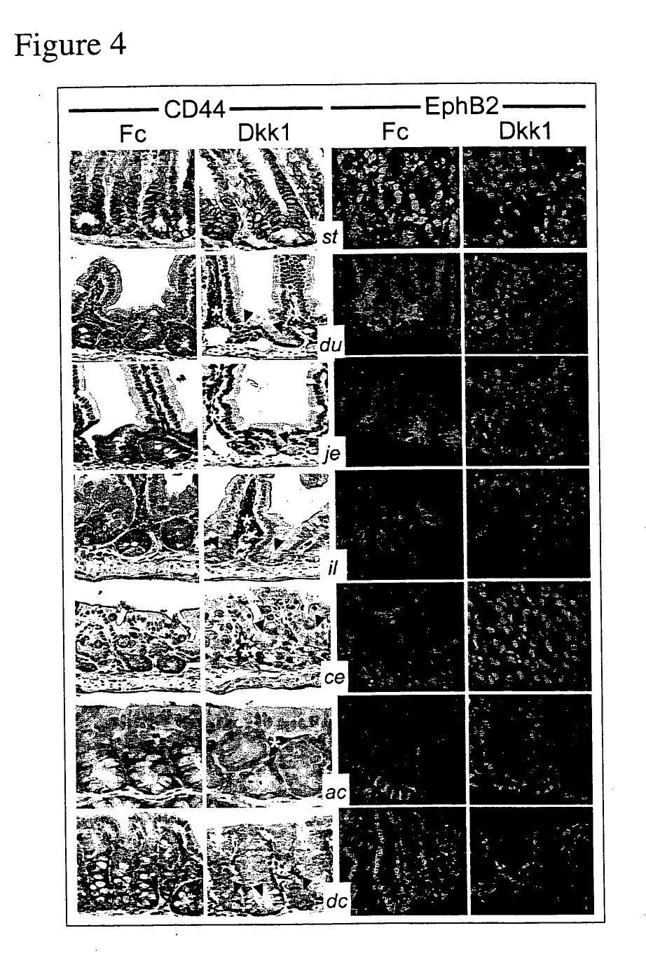

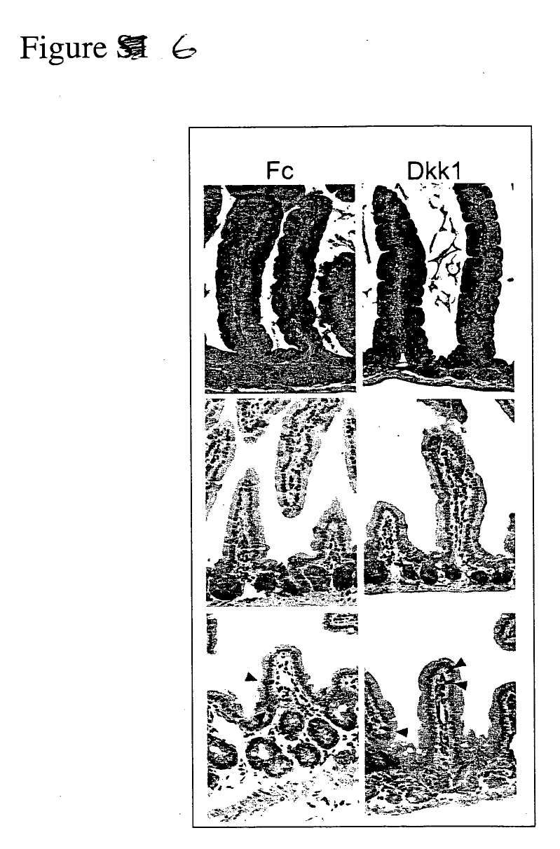

Confirming functional blockade of canonical Wnt signaling by Dkk1, the β-catenin/TCF target gene, CD44, was strongly and rapidly repressed within 2 days in duodenum and jejunum, with only nonepithelial lamina propria staining remaining (FIG. 4). Ad Dkk1 also potently repressed CD44 expression in ileum, despite the lack of gross architectural changes (FIG. 2). Epithelial CD44 expression was markedly reduced by Dkk1 in cecum and distal colon and partially reduced in proximal colon but was unaffected in stomach. Dkk1 also repressed the β-catenin/TCF target gene, EphB2, in duodenum, jejunum, ileum, cecum, and descending colon, with mild repression in ascending colon, and little to no repression in stomach (FIG. 4). In contrast, the magnitude or location of expression of epithelial differentiation markers for absorptive enterocytes or secretory lineages was not altered by Dkk1 expression (FIG. 6).

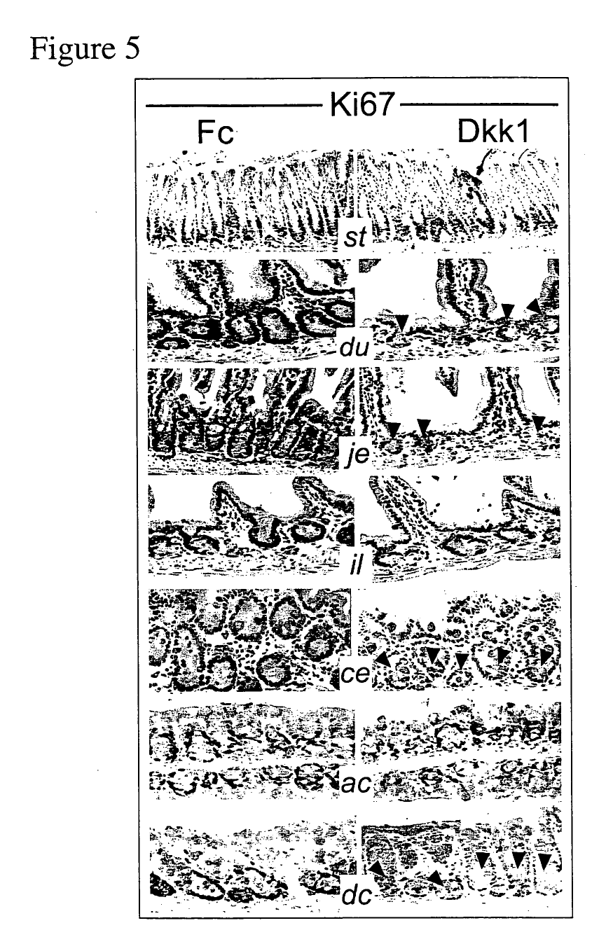

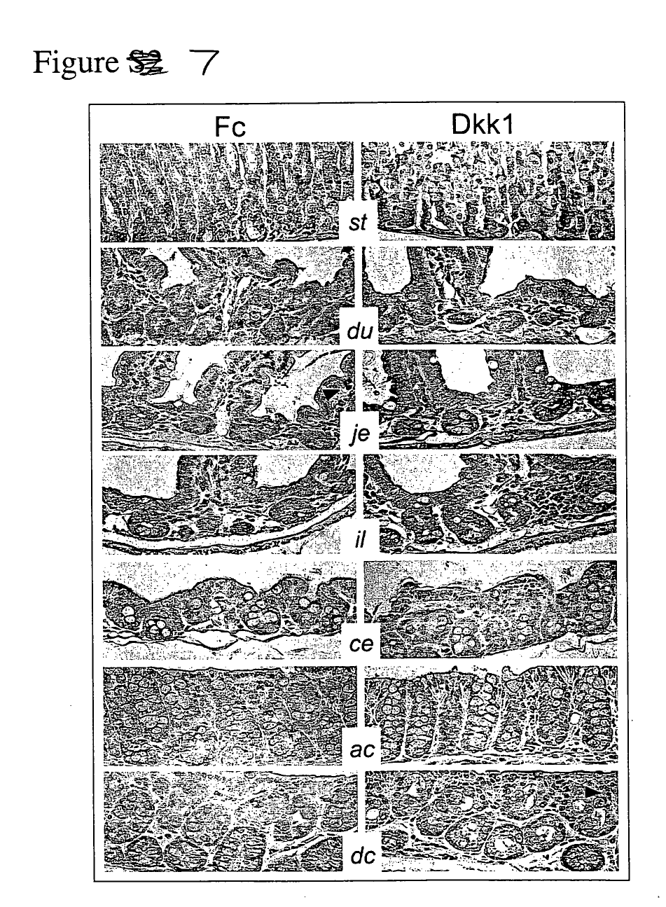

The proliferative status of the gastrointestinal epithelium in Ad Dkk1 mice was examined by immunohistochemistry for the S-phase marker, Ki67. Ad Dkk1 strikingly repressed enterocyte Ki67 immunoreactivity (>90%) within 2-4 days in duodenum and proximal jejunum, with any remnant crypts exhibiting diminished Ki67 staining and residual expression largely confined to nonepithelial cells of the lamina propria (FIG. 5). Proliferation in jejunum, along the proximal-distal axis, was progressively less affected by Ad Dkk1 to the extent that Ki67 staining in the ileum was not significantly inhibited by Ad Dkk1 (FIG. 5), despite effective repression of CD44 and EphB2 expression (FIG. 4). Epithelial Ki67 staining was also substantially reduced (70-80%) in cecum and descending colon, moderately reduced in ascending colon (60%), and not significantly affected in stomach. (FIG. 5). In contrast, TUNEL staining did not reveal increased apoptosis in either the proliferative crypts or differentiated villi/glands of the stomach, small intestine or colon (FIG. 7). In total, these data indicated that Dkk1 elicited stringent in vivo blockade of canonical Wnt signaling in both small intestine and colon, with repression of both Wnt target gene expression and epithelial proliferation in parallel.

We have achieved stringent, fully conditional and reversible inactivation of Wnt signaling in adult mice by adenoviral expression of the soluble Wnt inhibitor Dkk1, which functions as a pan-inhibitor of canonical Wnt signaling through interactions with the Wnt coreceptors, LRP5/6. The extensive Ad Dkk1 repression of proliferation and of -catenin/TCF target genes, as well as the progressive loss of villi and glands in small intestine, cecum, and colon to the point of mucosal ulceration, implicates the Wnt receptor complex and canonical Wnt signaling in maintenance of gene expression and architecture throughout the intestinal epithelium, which is consistent with, but much more extensive than, the mild reduction of villus number in Tcf4−/− mouse small intestine. The additional colon and cecum phenotypes observed in Dkk1 mice could result from either Dkk1 membrane-proximal interference with Wnt signaling versus membrane-distal effects in Tcf-4−/− animals, or from Tcf-3/Tcf-4 redundancy. Analogous mechanistic redundancy with non-Wnt- or non-Dkk1-sensitive pathways may underlie the observed proximal-distal phenotypic gradient in Ad Dkk1 small intestine (FIGS. 2 and 3), as well as the Dkk1 inhibition of Wnt target gene expression but not proliferation in ileum (FIGS. 4 and 5). Given the direct action of Dkk1 on the LRP/frizzled receptor complex, as opposed to the membrane-distal action of Tcf-4, the current data demonstrate Dkk1-sensitive Wnt signaling as essential for maintenance of both proliferation and architecture of the intestinal epithelium in adult animals.

The current data, using a distinct, fully conditional adenoviral approach, suggest a broad physiologic role for Wnt signaling in the adult gastrointestinal tract that is not restricted to the small bowel, but is a general property of the intestinal glandular epithelium, whether in small intestine or colon.

The finding of Wnt-dependent proliferation in the colon raises several therapeutic and pathophysiologic correlates. The ability of Ad Dkk1 to produce colitis suggests its potential utility as a novel inducible animal model of inflammatory bowel disease (IBD). The current data, in which Wnt blockade results in inhibition of colonic epithelial proliferation and progressive architectural degeneration, indicate that Wnt proteins represent essential growth factors for proliferative compartments of the large intestine.

The present data indicate a therapeutic role for Wnt proteins to encourage mucosal regeneration during IBD, perhaps as an adjunctive therapy to antiinflammatory or immunosuppressive therapy, by analogy to recently described growth factor-based IBD therapy by using epidermal growth factor. The investigation of nongastrointestinal phenotypes in lower dose Ad Dkk1 animals, adenoviral expression of other classes of soluble Wnt inhibitors, or the use of regeneration models, may prove informative and reinforce the use of soluble Wnt antagonists as a general strategy for conditional Wnt inhibition in adult animals.

The current data reveal a striking reliance on a single signaling pathway for the maintenance of both the proliferation and architecture of an adult organ and indicate that a strict dependence on canonical Wnt signaling is a general property of intestinal epithelium, whether in small intestine or colon. The present findings suggest that Wnt proteins represent primary candidates for the long-sought growth factor(s) responsible for maintenance of the adult intestinal epithelial stem cell niche, by analogy to the recent description of Wnt3a as a potent renewal factor for hematopoietic stem cells. The stringent requirement for Dkk1-sensitive Wnt signaling demonstrated herein recalls the requirement of hematopoietic precursors for growth factors such as erythropoietin and G-CSF and demonstrates the utility of therapeutic manipulation of Wnt pathways, agonistic or antagonistic, for disorders of the intestinal epithelium.

### EXAMPLE 2

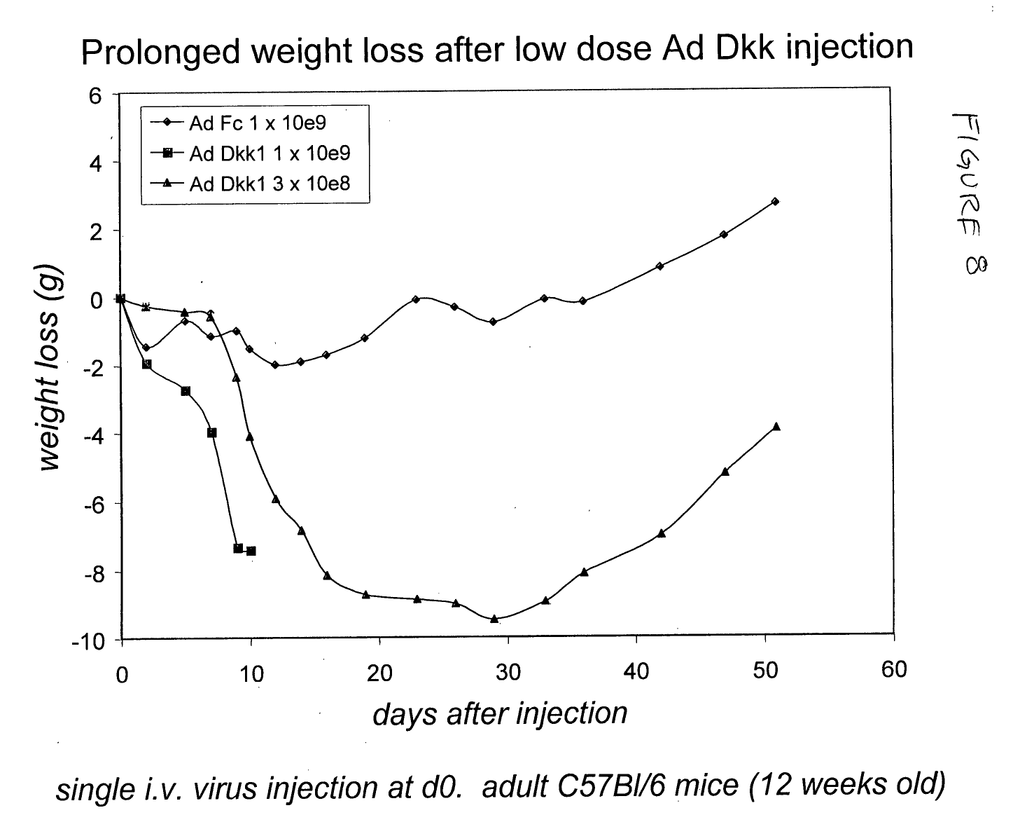

Adult C57BI/6 mice (12 weeks old) were given a single injection of the Ad DKK vector at 3×10⁸ pfu, or 10⁹ PFU. Weight was monitored for a period of greater than 50 days. As shown in FIG. 8, the treatment resulted in prolonged weight loss.

1. A method for the modulation of intestinal epithelial cell proliferation, the method comprising:
contacting intestinal epithelial cells with a modulator of wnt signaling.

2. A method of treating gastrointestinal diseases that compromise the intestinal epithelia, the method comprising:
administering to an individual suffering from said gastrointestinal disease, an agent that activates wnt signaling pathways.

3. The method according to claim 2, wherein said modulator of wnt signaling is a polypeptide.

4. The method according to claim 2, wherein said modulator of wnt signaling is a nucleic acid encoding a polypeptide.

5. The method according to claim 2, wherein said modulator of wnt signaling is a small organic molecule.

6. The method according to claim 2, wherein said modulator of wnt signaling is formulated with a pharmaceutically acceptable excipient.

7. The method according to claim 2, wherein said modulator of wnt signaling is administered orally.

8. The method according to claim 2, wherein said modulator of wnt signaling is is contained in a formulation that comprises an enteric coating.

9. A method of treating obesity, the method comprising:
administering to an individual suffering from obesity an inhibitor of wnt signaling, in an amount effective to diminish proliferation of intestinal epithelial cell proliferation.

10. The method according to claim 9, wherein said modulator of wnt signaling is a polypeptide.

11. The method according to claim 9, wherein said modulator of wnt signaling is a nucleic acid encoding a polypeptide.

12. The method according to claim 9, wherein said modulator of wnt signaling is a small organic molecule.

13. The method according to claim 9, wherein said modulator of wnt signaling is formulated with a pharmaceutically acceptable excipient.

14. The method according to claim 9, wherein said modulator of wnt signaling is administered orally.

15. The method according to claim 9, wherein said modulator of wnt signaling is is contained in a formulation that comprises an enteric coating.
